(19) United States
(12) Patent Application Publication
(10) Pub. No.: US 2024/0081338 A1
(43) Pub. Date: Mar. 14, 2024

(54) Microbial Inoculants and Methods

(71) Applicant: Ceragen Inc., Kitchener (CA)

(72) Inventors: Danielle Rose, Waterloo (CA); Ashley Stegelmeier, Kitchener (CA); Matthew Rose, Waterloo (CA); Ben Joris, Kitchener (CA); Erika Serravalle, Kitchener (CA); Michelle Drapeau, Kitchener (CA)

(21) Appl. No.: 18/453,741

(22) Filed: Aug. 22, 2023

Related U.S. Application Data
(60) Provisional application No. 63/400,799, filed on Aug. 25, 2022.
(60) Provisional application No. 63/452,849, filed on Mar. 17, 2023.

(51) Int. Cl. A01N 63/20; A01P 21/00

(52) U.S. Cl. CPC A01N 63/20; A01P 21/00

(57) ABSTRACT

In the present invention, microbial inoculants that promoted leafy green plant growth were developed using Rhodococcus erythropolis, Stenotrophomonas maltophilia, Serratia grimesii, and Pseudomonas toyotomiensis. The microbial inoculants include probiotics effective on their own and others effective in specific combinations as follows: Rhodococcus erythropolis; Rhodococcus erythropolis and Serratia grimesii; Serratia grimesii; Serratia grimesii and Pseudomonas toyotomiensis; Pseudomonas toyotomiensis and Stenotrophomonas maltophilia; Rhodococcus erythropolis, Pseudomonas toyotomiensis and Stenotrophomonas maltophilia; Rhodococcus erythropolis, Pseudomonas toyotomiensis, Stenotrophomonas maltophilia, and Serratia grimesii; Shinella zoogloeoides; and Rhodococcus erythropolis, Pseudomonas toyotomiensis, Stenotrophomonas maltophilia, Serratia grimesii and Shinella zoogloeoides. It was found that other probiotics were not effective on their own and there were other combinations that were not effective or inhibitory for plant growth.

### CROSS-REFERENCE TO RELATED APPLICATION

This application claims priority to United States Provisional Patent Application Nos. 63/400,799 filed Aug. 25, 2022 and 63/452,849 filed Mar. 17, 2023, the disclosures of which are hereby incorporated by reference in their entireties.

### SEQUENCE LISTING

The Sequence Listing associated with this application is filed in electronic format via Patent Center and is hereby incorporated by reference into the specification in its entirety. The name of the file containing the Sequence Listing is 2306509.xml. The size of the file is 12,736 bytes, and the file was created on Aug. 21, 2023.

### BACKGROUND OF THE INVENTION

### Field of the Invention

The present invention relates to microbial inoculants and more specifically to microbial inoculants that promoted leafy green plant growth either singly or in specific combinations.

### Description of Related Art

The plant microbiome is the community of bacteria, archaea, fungi, and viruses that live on, around, and inside of a plant.¹ Microbes play an important role in the health and wellbeing of plants, including helping plants respond better to environmental stresses.² These microbes can help provide nutrients for the plant, promote plant growth, and regulate hormone levels. For some plants it is known that microbial inoculants can increase crop yields by reducing negative stress effects such as leaf yellowing, leaf loss, and impaired plant growth. Microbial inoculants have also been shown to increase crop yields by improving the uptake or availability to the plant of nutrients, such as iron or phosphorus.³

Microbial inoculants are compositions comprising beneficial microbes that promote plant health. Microbial inoculants can help farmers significantly increase crop yields. However, bacterial species are specific to the type of plants that they can interact with,⁴⁻⁵ and many microbes cannot make the transition to soilless based growing systems. The nutritional composition of the plant root exudates contains a complex mixture of sugars, amino acids, hormones, antimicrobials, and vitamins.¹ The variation in root exudates between plants is one of the main drivers that determines which microorganisms can associate with the roots of each species.¹ By extension, previous research has determined that root colonization capacity does not predict the ability of an organism to survive in hydroponics systems.⁶ A study by Politz et al concluded that “In general, in vitro assays were not useful predictors of the relative performance of isolates in planta”, likely due to their methodology of using microorganisms obtained from soils to inoculate cucumbers in a hydroponics environment.⁷ When soil was added into a hydroponics system containing maize seedlings, a community shift in the types of organisms that can effectively survive on the roots was observed.⁸ The successful root colonizers were predominately shorter rods with simpler nutritional demands compared to the microbial community found in soil.

To date, research on plant growth promoting bacteria in lettuce has focused primarily on soil-based studies. Numerous researchers have demonstrated that meaningful yield increases in soil-based lettuce agriculture can be achieved using microbial inoculants.⁹⁻¹⁸ However, fewer studies have specifically focused on hydroponics lettuce research.¹⁹⁻²⁶ These studies provided the foundation that microbial inoculants can be effective in lettuce. However, their conclusions were often based on relatively small numbers of plant replicates, few candidate bacterial strains, or only tested a single bacterial strain instead of a consortium. There is currently no commercially available microbial inoculant designed and optimized specifically for hydroponic leafy greens.

### SUMMARY OF THE INVENTION

The present invention is a microbial inoculant for promoting leafy green plant growth comprising Rhodococcus erythropolis.

The present invention is a microbial inoculant for promoting leafy green plant growth comprising Rhodococcus erythropolis with a 98% nucleotide sequence similarity to the 16S region of SEQ ID NO:1.

The present invention is a microbial inoculant for promoting leafy green plant growth comprising Rhodococcus erythropolis and Serratia grimesii.

The present invention is a microbial inoculant for promoting leafy green plant growth comprising Serratia grimesii.

The present invention is a microbial inoculant for promoting leafy green plant growth comprising Serratia grimesii with a 98% nucleotide sequence similarity to the 16S region of SEQ ID NO:2. The present invention is a microbial inoculant for promoting leafy green plant growth comprising Serratia grimesii with a 98% nucleotide sequence similarity to the 16S region of SEQ ID NO:2 and Rhodococcus erythropolis with a 98% nucleotide sequence similarity to the 16S region of SEQ ID NO:1.

The present invention is a microbial inoculant for promoting leafy green plant growth comprising Serratia grimesii and Pseudomonas toyotomiensis.

The present invention is a microbial inoculant for promoting leafy green plant growth comprising Serratia grimesii with a 98% nucleotide sequence similarity to the 16S region of SEQ ID NO:2 and Pseudomonas toyotomiensis with a 98% nucleotide sequence similarity to the 16S region of SEQ ID NO:4.

The present invention is a microbial inoculant for promoting leafy green plant growth comprising Stenotrophomonas maltophilia and Pseudomonas toyotomiensis.

The present invention is a microbial inoculant for promoting leafy green plant growth comprising Stenotrophomonas maltophilia with a 98% nucleotide sequence similarity to the 16S region of SEQ ID NO:3 and Pseudomonas toyotomiensis with a 98% nucleotide sequence similarity to the 16S region of SEQ ID NO:4.

The present invention is a microbial inoculant for promoting leafy green plant growth comprising Stenotrophomonas maltophilia and Pseudomonas toyotomiensis and Rhodococcus erythropolis.

The present invention is a microbial inoculant for promoting leafy green plant growth comprising Stenotrophomonas maltophilia with a 98% nucleotide sequence similarity to the 16S region of SEQ ID NO:3 and Pseudomonas toyotomiensis with a 98% nucleotide sequence similarity to the 16S region of SEQ ID NO:4 and Rhodococcus erythropolis with a 98% nucleotide sequence similarity to the 16S region of SEQ ID NO:1.

The present invention is a microbial inoculant for promoting leafy green plant growth comprising Stenotrophomonas maltophilia and Pseudomonas toyotomiensis and Rhodococcus erythropolis and Serratia grimesii.

The present invention is a microbial inoculant for promoting leafy green plant growth comprising Stenotrophomonas maltophilia with a 98% nucleotide sequence similarity to the 16S region of SEQ ID NO:3 and Pseudomonas toyotomiensis with a 98% nucleotide sequence similarity to the 16S region of SEQ ID NO:4 and Rhodococcus erythropolis with a 98% nucleotide sequence similarity to the 16S region of SEQ ID NO:1 and Serratia grimesii with a 98% nucleotide sequence similarity to the 16S region of SEQ ID NO:2.

The present invention is a microbial inoculant for promoting leafy green plant growth comprising Shinella zoogloeoides.

The present invention is a microbial inoculant for promoting leafy green plant growth comprising Shinella zoogloeoides with a 98% nucleotide sequence similarity to the 16S region of SEQ ID NO:5.

The present invention is a microbial inoculant for promoting leafy green plant growth comprising Stenotrophomonas maltophilia and Pseudomonas toyotomiensis and Rhodococcus erythropolis and Serratia grimesii and Shinella zoogloeoides.

The present invention is a microbial inoculant for promoting leafy green plant growth comprising Stenotrophomonas maltophilia with a 98% nucleotide sequence similarity to the 16S region of SEQ ID NO:3 and Pseudomonas toyotomiensis with a 98% nucleotide sequence similarity to the 16S region of SEQ ID NO:4 and Rhodococcus erythropolis with a 98% nucleotide sequence similarity to the 16S region of SEQ ID NO:1 and Serratia grimesii with a 98% nucleotide sequence similarity to the 16S region of SEQ ID NO:2 and Shinella zoogloeoides with a 98% nucleotide sequence similarity to the 16S region of SEQ ID NO:5.

The present invention is any one of the above microbial inoculants in which the leafy green plant is Lactuca sativa.

The present invention is the use of any one of the above microbial inoculants in one of the growing systems of hydroponic, aeroponic, soilless or soil based.

The present invention is a method for promoting plant growth comprising applying a composition of any one of the above microbial inoculants directly or through a water supply to the roots of the leafy green plants in an amount that provides for positive benefits relative to a control leafy green plant that does not receive an application of said microbial inoculant.

### BRIEF DESCRIPTION OF THE DRAWINGS

FIG. 1 is a chart comparing the average percent yield increases of hydroponic lettuce that received a microbial inoculant of Rhodococcus erythropolis compared to control plants that received no inoculant, in a first embodiment of the present invention versus other inoculants.

FIG. 2 is a chart comparing the average percent yield increases of hydroponic lettuce that received a microbial inoculant of Serratia grimesii in a second embodiment of the present invention, and a microbial inoculant of a combination of Pseudomonas toyotomiensis and Serratia grimesii in a third embodiment of the present invention, versus control plants that received no inoculant.

FIG. 3 is a chart comparing the average percent yield increases of hydroponic lettuce that received a microbial inoculant of the second embodiment of the invention of Serratia grimesii, and a fourth embodiment of the present invention of a combination of Stenotrophomonas maltophilia and Pseudomonas toyotomiensis, and a fifth embodiment of the present invention of a combination of Serratia grimesii and Rhodococcus erythropolis, compared to control plants that received no inoculant.

FIG. 4 is a chart comparing the average percent yield increases of hydroponics lettuce that received a microbial inoculant of the first embodiment of the present invention of Rhodococcus erythropolis, the fifth embodiment of the present invention of a combination of Serratia grimesii and Rhodococcus erythropolis, a sixth embodiment of the present invention of Rhodococcus erythropolis, Pseudomonas toyotomiensis, and Stenotrophomonas maltophilia, and a seventh embodiment of the present invention of Rhodococcus erythropolis, Serratia grimesii, Stenotrophomonas maltophilia, and Pseudomonas toyotomiensis, compared to control plants that received no inoculant.

FIG. 5 is a box plot of the average fresh lettuce yields for a hydroponic lettuce inoculant trial comparing the control plants which received no microbial inoculant to the seventh embodiment of the present invention (Consortium 7) which is a combination of Rhodococcus erythropolis, Serratia grimesii, Stenotrophomonas maltophilia, and Pseudomonas toyotomiensis, an eighth embodiment of the present invention (Isolate 11) which is a pure culture of Shinella zoogloeoides, and a ninth embodiment of the present invention (Consortium 8) which is a combination of Rhodococcus erythropolis, Serratia grimesii, Stenotrophomonas maltophilia, Pseudomonas toyotomiensis, and Shinella zoogloeoides.

### DESCRIPTION OF THE INVENTION

The present invention is a microbial inoculant for increasing leafy green growth. In a first embodiment of the present invention, the microbial inoculant comprises Rhodococcus erythropolis.

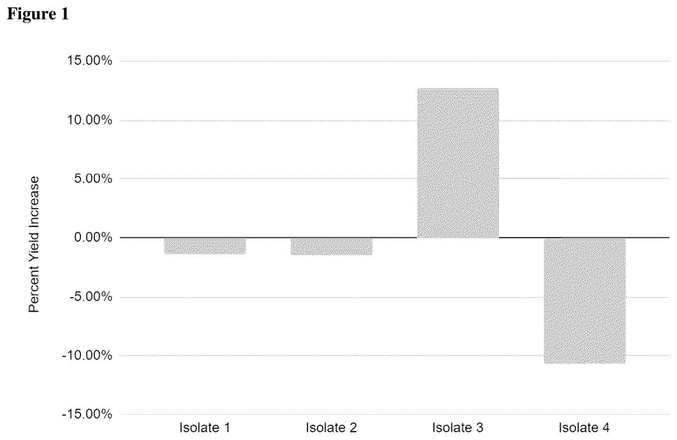

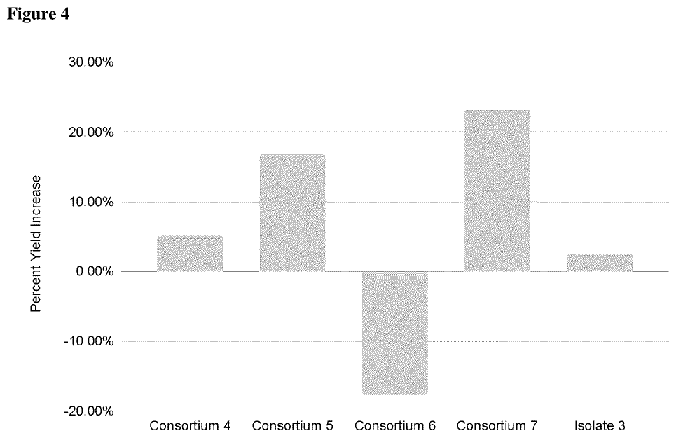

As set out in FIG. 1, Isolate 3 is a pure culture of Rhodococcus erythropolis. FIG. 1 shows the average percent yield increases for a hydroponic lettuce inoculant trial in comparison to the control plants that received no inoculant. Isolate 1 comprises a pure culture of Pseudomonas putida. Isolate 2 comprises a pure culture of Curvibacter putative. Isolate 4 comprises a pure culture of Xylophilus rhododendri. The microbial inoculant of Rhodococcus erythropolis is the only one to demonstrate increased yields in hydroponic lettuce. The increased yields from a microbial inoculant of Rhodococcus erythropolis is also demonstrated in FIG. 4.

In a second embodiment of the present invention, the microbial inoculant is Serratia grimesii. In a third embodiment of the present invention, the microbial inoculant is a combination of Pseudomonas toyotomiensis and Serratia grimesii.

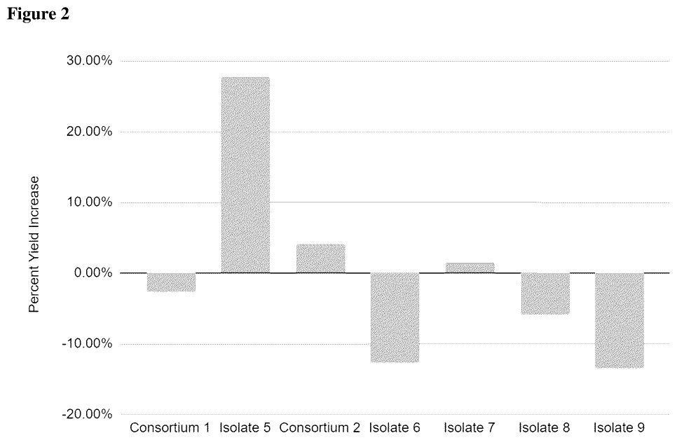

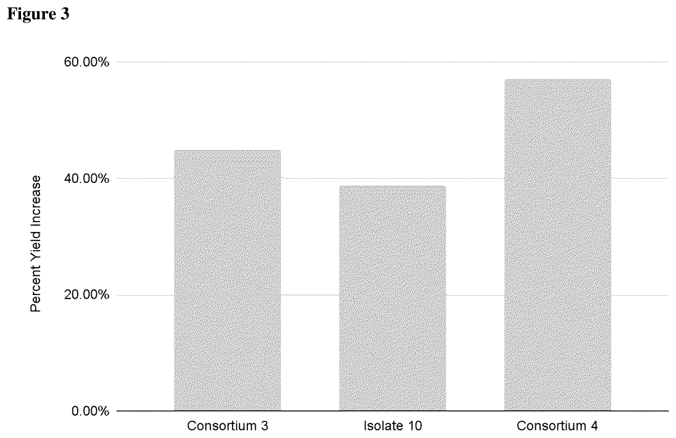

As set out in FIG. 2, Isolate 5 comprises a pure culture of Serratia grimesii and Consortium 2 comprises a combination of Pseudomonas toyotomiensis and Serratia grimesii. FIG. 2 shows the average percent yield increases for a hydroponic lettuce inoculant trial in comparison to the control plants that received no inoculant. Consortium 1 comprises a combination of Stenotrophomonas maltophilia and Serratia grimesii. Isolate 6 comprises a pure culture of Pseudomonas rhizophila. Isolate 7 is a pure culture of Pseudomonas zarinae. Isolate 8 is a pure culture of Pseudarthrobacter psychrotolerans. Isolate₉ comprises a pure culture of Paenarthrobacter aurescens. The microbial inoculant comprising Serratia grimesii and the microbial inoculant comprising the combination of Pseudomonas toyotomiensis and Serratia grimesii are the only ones to demonstrate significantly increased yields in hydroponic lettuce. The increased yields from a microbial inoculant of Serratia grimesii is also demonstrated in FIG. 3.

In a fourth embodiment of the present invention, the microbial inoculant is a combination of Stenotrophomonas maltophilia and Pseudomonas toyotomiensis. In a fifth embodiment of the present invention, the microbial inoculant is a combination of Serratia grimesii and Rhodococcus erythropolis.

FIG. 3 shows the average percent yield increases for a hydroponic lettuce inoculant trial in comparison to the control plants that received no inoculant. Consortium 3 comprises a combination of Stenotrophomonas maltophilia, and Pseudomonas toyotomiensis. Isolate 10 comprises a pure culture of Serratia grimesii. Consortium 4 comprises a combination of Serratia grimesii and Rhodococcus erythropolis. The increased yields from a microbial inoculant comprising a combination of Serratia grimesii and Rhodococcus erythropolis is also demonstrated in FIG. 4.

In a sixth embodiment of the present invention, the microbial inoculant is a combination of Rhodococcus erythropolis, Pseudomonas toyotomiensis, and Stenotrophomonas maltophilia. In a seventh embodiment of the present invention, the microbial inoculant comprises a combination of Rhodococcus erythropolis, Serratia grimesii, Stenotrophomonas maltophilia, and Pseudomonas toyotomiensis.

As set out in FIG. 4, Consortium 5 comprises a combination of Rhodococcus erythropolis, Pseudomonas toyotomiensis, and Stenotrophomonas maltophilia and Consortium 7 comprises a combination of Rhodococcus erythropolis, Serratia grimesii, Stenotrophomonas maltophilia, and Pseudomonas toyotomiensis. FIG. 4 shows the average percent yield increases for a hydroponic lettuce inoculant trial in comparison to the control plants that received no inoculant. Consortium 4 comprises a combination of Serratia grimesii and Rhodococcus erythropolis. Consortium 6 comprises a combination of Rhodococcus erythropolis and Pseudomonas stutzeri. Isolate 3 comprises a pure culture of Rhodococcus erythropolis. Consortium 6 is the only microbial inoculant not to show increased yield.

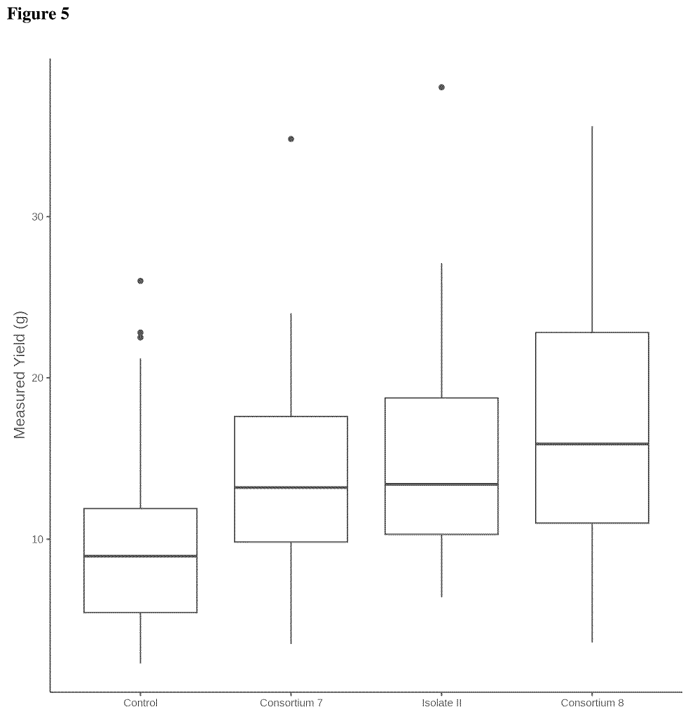

As shown in FIG. 5, the seventh embodiment of the present invention (Consortium 7) which is a combination of Rhodococcus erythropolis, Serratia grimesii, Stenotrophomonas maltophilia, and Pseudomonas toyotomiensis shows improved weight yields compared to the average fresh lettuce yields for a hydroponic lettuce which received no microbial inoculant, in alignment with the percent yield increase shown for the seventh embodiment in FIG. 4. As also shown in FIG. 5, improved weight yields over control were also found with an eighth embodiment of the present invention (Isolate 11) which is a pure culture of Shinella zoogloeoides, and with a ninth embodiment of the present invention (Consortium 8) which is a combination of Rhodococcus erythropolis, Serratia grimesii, Stenotrophomonas maltophilia, Pseudomonas toyotomiensis, and Shinella zoogloeoides.

None of the bacterial species of Rhodococcus erythropolis, Stenotrophomonas maltophilia, Serratia grimesii, Pseudomonas toyotomiensis, and Shinella zoogloeoides in the microbial inoculants of the present invention were reported in scientific peer reviewed literature as plant growth promoters in hydroponic systems. The present invention is the use of these microbial inoculants comprising Rhodococcus erythropolis, Stenotrophomonas maltophilia, Serratia grimesii Pseudomonas toyotomiensis, and Shinella zoogloeoides for hydroponic lettuce or other leafy plants, including kale, arugula, and spinach.

To develop the microbial inoculants of the present invention lettuce roots were processed to obtain root associated microbes. The bacteria were purified to single isolates. In total, 105 unique lettuce isolates were obtained. These isolates were not genetically modified.

The bacterial strains were screened for a wide range of biochemical and genetic characteristics that are known to increase agricultural yields. Additionally, bacterial strains were tested against a set of nine antibiotics covering the major antibiotic classes. Bacteria with high levels of multi-drug resistance were excluded from downstream testing.

The 105 bacterial strains underwent whole genome sequencing using Oxford Nanopore MinION technology. Using an in-house bioinformatics pipeline, their genes were analyzed for plant growth promoting capabilities. Bacteria that had both low antibiotic resistance and high levels of favourable genetic traits and biochemical lab results were tested in hydroponics lettuce to measure increases in growth rate.

Of the 105 candidate bacteria strains, only 21 indicated potential to contribute to plant growth promotion. These 21 strains were subsequently tested in a hydroponics grow tent versus control lettuce that was grown without probiotics. Of the 21 promising strains, only 10 resulted in increased lettuce biomass. These 10 strains were mixed into a range of combinations to further increase plant yields.

Extensive grow tent testing on over 8000 plants was required to determine both the individual strains and consortiums that consistently increased crop yields. The host plant specificity required a complexity of consortium formulations. A probiotic mixture that synergistically utilizes the beneficial contributions from several separate plant growth promoters is the result of a year of scientific pursuit. FIGS. 1 to 5 on lettuce biomass yield data shows a benefit of the present invention.

In use, the microbial inoculants of the present invention are applied to the root zone of the plant either through direct application or by addition to the watering system. In addition to the bacterial cells, the microbial inoculant may also comprise bacterial growth medium and stabilizing agents to increase shelf life.

The trials set out in the figures use the leafy green plant of lettuce, namely Lactuca sativa, grown hydroponically. It will be understood that the microbial inoculants may be used on other leafy green plants that are grown hydroponically. Hydroponics growers often have multiple species of leafy green varieties growing concurrently within their farming setup. Examples of other leafy green plants that will receive the positive benefits of the present invention's microbial inoculants are kale, arugula, and spinach.

The microbial inoculants of the present invention were developed to be compatible with use in multiple different hydroponics growing systems and hydroponics substrates (e.g. sponges, coco coir, rockwool, and peat moss). The microbial inoculants of the present invention may be used with leafy green plants in hydroponic based growing systems, aeroponic based growing systems, and other soilless based growing systems, since the microbes are root associated and substrate independent. The microbial inoculant can be applied to whatever medium the leafy green plants are growing in or be added to the water supplying the plants so that the microbial inoculant can contact the roots and begin colonization.

The microbial inoculants of the present invention include probiotics with identical or nearly identical 16S gene sequences as in the sequence listings. Sequence listing 1 is that of Rhodococcus erythropolis, sequence listing 2 is that of Serratia grimesii, sequence listing 3 is that of Stenotrophomonas maltophilia, sequence listing 4 is that of Pseudomonas toyotomiensis, and sequence listing 5 is that of Shinella zoogloeoides.

The present invention includes microbial inoculants comprising a probiotic or combination of probiotics identical or nearly identical or 98% identical to the respective sequence listings 1 to 5 of:
Rhodococcus erythropolis;
Rhodococcus erythropolis and Serratia grimesii;
Serratia grimesii;
Serratia grimesii and Pseudomonas toyotomiensis;
Pseudomonas toyotomiensis and Stenotrophomonas maltophilia;
Rhodococcus erythropolis, Pseudomonas toyotomiensis and Stenotrophomonas maltophilia;
Rhodococcus erythropolis, Pseudomonas toyotomiensis, Stenotrophomonas maltophilia and Serratia grimesii
Shinella zoogloeoides; and
Rhodococcus erythropolis, Pseudomonas toyotomiensis, Stenotrophomonas maltophilia, Serratia grimesii and Shinella zoogloeoides.

Probiotics with similar or corresponding 16S gene sequences, including 98% similarity, may generally be expected to have similar characteristics and similar beneficial effects on leafy green plants. A higher percent identity indicates a closer relationship between two or more microbial strains or isolates. A high degree of similarity or relatedness between two closely related microbial strains or isolates may also indicate a similar ability to impart the same or similar positive agricultural trait or benefit to a leafy green plant. A 16S gene sequence of sequence listing 1 to 5 corresponds to a 16S gene sequence of a second microbial strain if the two stains optimally align.

Probiotic cultures of the microbial inoculants of the present invention may be prepared using any standard or known fermentation techniques, or developed. Those skilled in microbial growth parameters are capable of determining the appropriate nutrients and conditions, including media and incubation conditions. Similarly, the microbial inoculants may comprise various formulations to support the maintenance and stability of the probiotic. Microbial inoculant compositions may comprise a pure or substantially pure population or culture of the microbial strain or isolate, or combination described herein. The term microbial inoculant refers to the combination of microbial strain or isolate combined with one or more other ingredients to form a composition that can be applied to the media or into the water for leafy green plants. The microbial inoculants of the present invention comprise an effective amount of the probiotic or combination of probiotics to impart a positive benefit on a leafy green plant. Compositions of the microbial inoculant include any agricultural carrier or other materials that benefit the probiotic culture and/or benefit or have no adverse effect on the leafy green plant.

While embodiments of the invention have been described in the detailed description, the scope of the claims should not be limited by the preferred embodiments set forth in the examples, but should be given the broadest interpretation consistent with the description as a whole.

SEQUENCE LISTINGS

SEQ ID NO: 1 Rhodococcus erythropolis
TTTGATCCTGGCTCAGGACGAACGCTGGCGGCGTGCTTAACACATGCAAG
TCGAGCGGTAAGGCCTTTCGGGGTACACGAGCGGCGAACGGGTGAGTAAC
ACGTGGGTGATCTGCCCTGCACTTCGGGATAAGCCTGGGAAACTGGGTCT
AATACCGGATATGACCTCCTATCGCATGGTGGGTGGTGGAAAGATTTATC
GGTGCAGGATGGGCCCGCGGCCTATCAGCTTGTTGGTGGGGTAATGGCCT
ACCAAGGCGACGACGGGTAGCCGACCTGAGAGGGTGACCGGCCACACTGG
GACTGAGACACGGCCCAGACTCCTACGGGAGGCAGCAGTGGGGAATATTG
CACAATGGGCGAAAGCCTGATGCAGCGACGCCGCGTGAGGGATGACGGCC
TTCGGGTTGTAAACCTCTTTCAGCAGGGACGAAGCGCAAGTGACGGTACC
TGCAGAAGAAGCACCGGCTAACTACGTGCCAGCAGCCGCGGTAATACGTA
GGGTGCAAGCGTTGTCCGGAATTACTGGGCGTAAAGAGTTCGTAGGCGGT
TTGTCGCGTCGTTTGTGAAAACCAGCAGCTCAACTGCTGGCTTGCAGGCG
ATACGGGCAGACTTGAGTACTGCAGGGGAGACTGGAATTCCTGGTGTAGC
GGTGAAATGCGCAGATATCAGGAGGAACACCGGTGGCGAAGGCGGGTCTC
TGGGCAGTAACTGACGCTGAGGAACGAAAGCGTGGGTAGCGAACAGGATT
AGATACCCTGGTAGTCCACGCCGTAAACGGTGGGCGCTAGGTGTGGGTTC
CTTCCACGGAATCCGTGCCGTAGCTAACGCATTAAGCGCCCCGCCTGGGG
AGTACGGCCGCAAGGCTAAAACTCAAAGGAATTGACGGGGGCCCGCACAA
GCGGCGGAGCATGTGGATTAATTCGATGCAACGCGAAGAACCTTACCTGG
GTTTGACATATACCGGAAAGCTGCAGAGATGTGGCCCCCCTTGTGGTCGG
TATACAGGTGGTGCATGGCTGTCGTCAGCTCGTGTCGTGAGATGTTGGGT
TAAGTCCCGCAACGAGCGCAACCCCTATCTTATGTTGCCAGCACGTTATG
GTGGGGACTCGTAAGAGACTGCCGGGGTCAACTCGGAGGAAGGTGGGGAC
GACGTCAAGTCATCATGCCCCTTATGTCCAGGGCTTCACACATGCTACAA
TGGCCAGTACAGAGGGCTGCGAGACCGTGAGGTGGAGCGAATCCCTTAAA
GCTGGTCTCAGTTCGGATCGGGGTCTGCAACTCGACCCCGTGAAGTCGGA
GTCGCTAGTAATCGCAGATCAGCAACGCTGCGGTGAATACGTTCCCGGGC
CTTGTACACACCGCCCGTCACGTCATGAAAGTCGGTAACACCCGAAGCCG
GTGGCTTAACCCCTTGTGGGAGGGAGCCGTCGAAGGTGGGATCGGCGATT
GGGACGAAGTCGTAACAAGGTAGCCGTACCGGAAGGTGCGGCTGGATCAC
CTCC
SEQ ID NO: 2 Serratia grimesii
TTGAAGAGTTTGATCATGGCTCAGATTGAACGCTGGCGGCAGGCCTAAC
ACATGCAAGTCGAGCGGTAGCACAAGAGAGCTTGCTCTCTGGGTGACGA
GCGGCGGACGGGTGAGTAATGTCTGGGAAACTGCCTGATGGAGGGGGAT
AACTACTGGAAACGGTAGCTAATACCGCATAACGTCTTCGGACCAAAGT
GGGGGACCTTCGGGCCTCACGCCATCAGATGTGCCCAGATGGGATTAGC
TAGTAGGTGGGGTAATGGCTCACCTAGGCGACGATCCCTAGCTGGTCTG
AGAGGATGACCAGCCACACTGGAACTGAGACACGGTCCAGACTCCTACG
GGAGGCAGCAGTGGGGAATATTGCACAATGGGCGCAAGCCTGATGCAGC
CATGCCGCGTGTGTGAAGAAGGCCTTCGGGTTGTAAAGCACTTTCAGCG
AGGAGGAAGGGTAGTGTGTTAATAGCACATTGCATTGACGTTACTCGCA
GAAGAAGCACCGGCTAACTCCGTGCCAGCAGCCGCGGTAATACGGAGGG
TGCAAGCGTTAATCGGAATTACTGGGCGTAAAGCGCACGCAGGCGGTTT
GTTAAGTCAGATGTGAAATCCCCGCGCTTAACGTGGGAACTGCATTTGA
AACTGGCAAGCTAGAGTCTTGTAGAGGGGGGTAGAATTCCAGGTGTAGC
GGTGAAATGCGTAGAGATCTGGAGGAATACCGGTGGCGAAGGCGGCCCC
CTGGACAAAGACTGACGCTCAGGTGCGAAAGCGTGGGGAGCAAACAGGA
TTAGATACCCTGGTAGTCCACGCTGTAAACGATGTCGACTTGGAGGTTG
TGCCCTTGAGGCGTGGCTTCCGGAGCTAACGCGTTAAGTCGACCGCCTG
GGGAGTACGGCCGCAAGGTTAAAACTCAAATGAATTGACGGGGGCCCGC
ACAAGCGGTGGAGCATGTGGTTTAATTCGATGCAACGCGAAGAACCTTA
CCTACTCTTGACATCCAGAGAATTCGCTAGAGATAGCTTAGTGCCTTCG
GGAACTCTGAGACAGGTGCTGCATGGCTGTCGTCAGCTCGTGTTGTGAA
ATGTTGGGTTAAGTCCCGCAACGAGCGCAACCCTTATCCTTTGTTGCCA
GCACGTAATGGTGGGAACTCAAAGGAGACTGCCGGTGATAAACCGGAGG
AAGGTGGGGATGACGTCAAGTCATCATGGCCCTTACGAGTAGGGCTACA
CACGTGCTACAATGGCGTATACAAAGAGAAGCGAACTCGCGAGAGCAAG
CGGACCTCATAAAGTACGTCGTAGTCCGGATCGGAGTCTGCAACTCGAC
TCCGTGAAGTCGGAATCGCTAGTAATCGTAGATCAGAATGCTACGGTGA
ATACGTTCCCGGGCCTTGTACACACCGCCCGTCACACCATGGGAGTGGG
TTGCAAAAGAAGTAGGTAGCTTAACCTTCGGGAGGGCGCTTACCACTTT
GTGATTCATGACTGGGGTGAAGTCGTAACAAGGTAACCGTAGGGGAACC
TGCGGTTGGATCACCTCCT
SEQ ID NO: 3 Stenotrophomonas maltophilia
TGAAGAGTTTGATCCTGGCTCAGAGTGAACGCTGGCGGTAGGCCTAACAC
ATGCAAGTCGAACGGCAGCACAGAGGAGCTTGCTCCTTGGGTGGCGAGTG
GCGGACGGGTGAGGAATACATCGGAATCTACTTTTTCGTGGGGGATAACG
TAGGGAAACTTACGCTAATACCGCATACGACCTACGGGTGAAAGCAGGGG
ATCTTCGGACCTTGCGCGATTGAATGAGCCGATGTCGGATTAGCTAGTTG
GCGGGGTAAAGGCCCACCAAGGCGACGATCCGTAGCTGGTCTGAGAGGAT
GATCAGCCACACTGGAACTGAGACACGGTCCAGACTCCTACGGGAGGCAG
CAGTGGGGAATATTGGACAATGGGCGCAAGCCTGATCCAGCCATACCGCG
TGGGTGAAGAAGGCCTTCGGGTTGTAAAGCCCTTTTGTTGGGAAAGAAAT
CCAGCCGGCTAATACCTGGTTGGGATGACGGTACCCAAAGAATAAGCACC
GGCTAACTTCGTGCCAGCAGCCGCGGTAATACGAAGGGTGCAAGCGTTAC
TCGGAATTACTGGGCGTAAAGCGTGCGTAGGTGGTTATTTAAGTCCGTTG
TGAAAGCCCTGGGCTCAACCTGGGAACTGCAGTGGATACTGGATGACTAG
AATGTGGTAGAGGGTAGCGGAATTCCTGGTGTAGCAGTGAAATGCGTAGA
GATCAGGAGGAACATCCATGGCGAAGGCAGCTACCTGGACCAACATTGAC
ACTGAGGCACGAAAGCGTGGGGAGCAAACAGGATTAGATACCCTGGTAGT
CCACGCCCTAAACGATGCGAACTGGATGTTGGGTGCAATTTGGCACGCAG
TATCGAAGCTAACGCGTTAAGTTCGCCGCCTGGGGAGTACGGTCGCAAGA
CTGAAACTCAAAGGAATTGACGGGGGCCCGCACAAGCGGTGGAGTATGTG
GTTTAATTCGATGCAACGCGAAGAACCTTACCTGGCCTTGACATGTCGAG
AACTTTCCAGAGATGGATGGGTGCCTTCGGGAACTCGAACACAGGTGCTG
CATGGCTGTCGTCAGCTCGTGTCGTGAGATGTTGGGTTAAGTCCCGCAAC
GAGCGCAACCCTTGTCCTTAGTTGCCAGCACGTAATGGTGGGAACTCTAA
GGAGACCGCCGGTGACAAACCGGAGGAAGGTGGGGATGACGTCAAGTCAT
CATGGCCCTTACGGCCAGGGCTACACACGTACTACAATGGTAGGGACAGA
GGGCTGCAAGCCGGCGACGGTAAGCCAATCCCAGAAACCCTATCTCAGTC
CGGATTGGAGTCTGCAACTCGACTCCATGAAGTCGGAATCGCTAGTAATC
GCAGATCAGCATTGCTGCGGTGAATACGTTCCCGGGCCTTGTACACACCG
CCCGTCACACCATGGGAGTTTGTTGCACCAGAAGCAGGTAGCTTAACCTT
CGGGAGGGCGCTTGCCACGGTGTGGCCGATGACTGGGGTGAAGTCGTAAC
AAGGTAGCCGTATCGGAAGGTGCGGCTGGATCACCTCCTT
SEQ ID NO: 4 Pseudomonas toyotomiensis
TGAAGAGTTTGATCATGGCTCAGATTGAACGCTGGCGGCAGGCCTAACAC
ATGCAAGTCGAGCGGATGAGGGGAGCTTGCTCCCTGATTTAGCGGCGGAC
GGGTGAGTAATGCCTAGGAATCTGCCTAGTGGTGGGGGATAACGTTCCGA
AAGGAACGCTAATACCGCATACGTCCTACGGGAGAAAGCGGGGGATCTTC
GGACCTCGCGCCATTAGATGAGCCTAGGTCGGATTAGCTAGTTGGTGAGG
TAAAGGCTCACCAAGGCGACGATCCGTAACTGGTCTGAGAGGATGATCAG
TCACACTGGAACTGAGACACGGTCCAGACTCCTACGGGAGGCAGCAGTGG
GGAATATTGGACAATGGGCGAAAGCCTGATCCAGCCATGCCGCGTGTGTG
AAGAAGGTCTTCGGATTGTAAAGCACTTTAAGTTGGGAGGAAGGGTATTC
ACCTAATACGTGAGTATTTTGACGTTACCGACAGAATAAGCACCGGCTAA
CTTCGTGCCAGCAGCCGCGGTAATACGAAGGGTGCAAGCGTTAATCGGAA
TTACTGGGCGTAAAGCGCGCGTAGGTGGTTCGTTAAGTTGGATGTGAAAG
CCCCGGGCTCAACCTGGGAACTGCATCCAAAACTGGCGAGCTAGAGTACG
GTAGAGGGTGGTGGAATTTCCTGTGTAGCGGTGAAATGCGTAGATATAGG
AAGGAACACCAGTGGCGAAGGCGACCACCTGGACTGATACTGACACTGAG
GTGCGAAAGCGTGGGGAGCAAACAGGATTAGATACCCTGGTAGTCCACGC
CGTAAACGATGTCAACTAGCCGTTGGAATCCTTGAGATTTTAGTGGCGCA
GCTAACGCATTAAGTTGACCGCCTGGGGAGTACGGCCGCAAGGTTAAAAC
TCAAATGAATTGACGGGGGCCCGCACAAGCGGTGGAGCATGTGGTTTAAT
TCGAAGCAACGCGAAGAACCTTACCTGGCCTTGACATGCTGAGAACTTTC
CAGAGATGGATTGGTGCCTTCGGGAACTCAGACACAGGTGCTGCATGGCT
GTCGTCAGCTCGTGTCGTGAGATGTTGGGTTAAGTCCCGTAACGAGCGCA
ACCCTTGTCCTTAGTTACCAGCACGTTATGGTGGGCACTCTAAGGAGACT
GCCGGTGACAAACCGGAGGAAGGTGGGGATGACGTCAAGTCATCATGGCC
CTTACGGCCAGGGCTACACACGTGCTACAATGGTCGGTACAAAGGGTTGC
CAAGCCGCGAGGTGGAGCTAATCCCATAAAACCGATCGTAGTCCGGATCG
CAGTCTGCAACTCGACTGCGTGAAGTCGGAATCGCTAGTAATCGTGAATC
AGAATGTCACGGTGAATACGTTCCCGGGCCTTGTACACACCGCCCGTCAC
ACCATGGGAGTGGGTTGCTCCAGAAGTAGCTAGTCTAACCTTCGGGGGGA
CGGTTACCACGGAGTGATTCATGACTGGGGTGAAGTCGTAACAAGGTAGC
CGTAGGGGAACCTGCGGCTGGATCACCTCCT
SEQ ID NO: 5 Shinella zoogloeoides
TTTGATCCTGGCTCAGAACGAACGCTGGCGGCAGGCTTAACACATGCAAG
TCGAACGCCCCGCAAGGGGAGTGGCAGACGGGTGAGTAACGCGTGGGAAT
CTACCCATCTCTACGGAATAACTCAGGGAAACTTGTGCTAATACCGTATA
CGCCCTTCGGGGGAAAGATTTATCGGAGATGGATGAGCCCGCGTTGGATT
AGCTAGTTGGTGGGGTAAAGGCCTACCAAGGCGACGATCCATAGCTGGTC
TGAGAGGATGATCAGCCACATTGGGACTGAGACACGGCCCAAACTCCTAC
GGGAGGCAGCAGTGGGGAATATTGGACAATGGGCGCAAGCCTGATCCAGC
CATGCCGCGTGAGTGATGAAGGCCCTAGGGTTGTAAAGCTCTTTCACCGG
TGAAGATAATGACGGTAACCGGAGAAGAAGCCCCGGCTAACTTCGTGCCA
GCAGCCGCGGTAATACGAAGGGGGCTAGCGTTGTTCGGAATTACTGGGCG
TAAAGCGCACGTAGGCGGGTATTTAAGTCAGGGGTGAAATCCCAGAGCTC
AACTCTGGAACTGCCTTTGATACTGGGTACCTAGAGTATGGAAGAGGTAA
GTGGAATTCCGAGTGTAGAGGTGAAATTCGTAGATATTCGGAGGAACACC
AGTGGCGAAGGCGGCTTACTGGTCCATTACTGACGCTGAGGTGCGAAAGC
GTGGGGAGCAAACAGGATTAGATACCCTGGTAGTCCACGCCGTAAACGAT
GAATGTTAGCCGTCGGCATGCATGCATGTCGGTGGCGCAGCTAACGCATT
AAACATTCCGCCTGGGGAGTACGGTCGCAAGATTAAAACTCAAAGGAATT
GACGGGGGCCCGCACAAGCGGTGGAGCATGTGGTTTAATTCGAAGCAACG
CGCAGAACCTTACCAGCCCTTGACATGTCGGTCGCGGATTACAGAGATGT
TTTCCTTCAGTTAGGCTGGACCGAACACAGGTGCTGCATGGCTGTCGTCA
GCTCGTGTCGTGAGATGTTGGGTTAAGTCCCGCAACGAGCGCAACCCTCG
CCCTTAGTTGCCAGCATTCAGTTGGGCACTCTAAGGGGACTGCCGGTGAT
AAGCCGAGAGGAAGGTGGGGATGACGTCAAGTCCTCATGGCCCTTACGGG
CTGGGCTACACACGTGCTACAATGGTGGTGACAGTGGGCAGCGAGACAGC
GATGTCGAGCTAATCTCCAAAAGCCATCTCAGTTCGGATTGCACTCTGCA
ACTCGAGTGCATGAAGTTGGAATCGCTAGTAATCGCGGATCAGCATGCCG
CGGTGAATACGTTCCCGGGCCTTGTACACACCGCCCGTCACACCATGGGA
GTTGGTTTTACCCGAAGGCACTGCGCTAACCGCAAGGGGGCAGGTGACCA
CGGTAGGGTCAGCGACTGGGGTGAAGTCGTAACAAGGTAGCCGTAGGGGA
ACCTGCGGCTGGATCACCTCC

### REFERENCES

1. Turner, T. R., James, E. K., & Poole, P. S. (2013). The plant microbiome. Genome Biology, 14:209.
2. Glick, B. R. (2014). Bacteria with ACC deaminase can promote plant growth and help to feed the world. Microbiological Research, 169(1): 30-39.
3. Sammauria, R., Kumawat, S., Kumawat, P., Singh, J., & Jatwa, T. K. (2020). Microbial inoculants: potential tool for sustainability of agricultural production systems. Archives of Microbiology, 202: 677-693.
4. Drogue, B., Doré H., Borland, S., Wisniewski-Dye, & F. Prigent-Combaret, C. (2012). Which specificity in cooperation between phytostimulating rhizobacteria and plants? Research in Microbiology, 163(8): 500-510.
5. Sanchis-López, C., Cerna-Vargas, J. P., Santamaria-Hernando, S., Ramos, C., Krell, T., Rodriguez-Palenzuela, P., López-Solanilla, E., Huerta-Cepas, J., & Rodriguez-Hervaa, J. J. (2021). Prevalence and specificity of chemoreceptor profiles in plant associated bacteria. mSystems, 6(5): e00951-21.
6. Moralles, A., Garland, J. L., & Lim, D. V. (1996). Survival of potentially pathogenic human-associated bacteria in the rhizosphere of hydroponically grown wheat. FEMS Microbiology Ecology, 20(1996): 155-162.
7. Paulitz, T. C., Zhou, T., & Rankin, L. (1992). Selection of rhizosphere bacteria for biological control of Pythium aphanidermatum on hydroponically grown cucumber. Biological Control, 2(3): 226-237.
8. Schonwitz, R., & Ziegler, H. (1986). Quantitative and qualitative aspects of a developing rhizosphere microflora of hydroponically grown maize seedling. Journal of Plant Nutrition and Soil Science, 149: 623-634.
9. Sahin, U., Ekinci, M., Kiziloglu, F. M., Yildirim, E., Turan, M., Kotan, R., & Ors, S. (2015). Ameliorative effects of plant growth promoting bacteria on water-yield relationships, growth, and nutrient uptake of lettuce plants under different irrigation levels. Hortscience, 50(9): 1379-1386.
10. Cipriano., M. A. P., Lupatini, M., Lopes-Santos, L., da Silva, M. J., Roesch, L. F. W., Destefano, S. A. L., Freitas, S. S., & Kuramae, E. E. (2016). Lettuce and rhizosphere microbiome responses to growth promoting Pseudomonas species under field conditions. FEMS Microbiology Ecology, 92(12): fiw197.
11. Szczech, M., Szafirowska, A., Kowalczyk, W., Szwejda-Grzybowska, J., Wlodarek, A., & Maciorowski, R. (2016). The effect of plant growth promoting bacteria on transplants growth and lettuce yield in organic production. Journal of Horticultural Research, 24(2): 101-107.
12. Ferreira, J. T. P., Santos, T. M. C., Albuquerque, L. S., Santos, J. V., Cardoso Filho, J. A. C., & Ramalho Neto, C. E. (2011). Isolation and selection of growth-promoting bacteria of the genus Bacillus and its effect on two varieties of lettuce (Lactuca sativa L.). International Research Journal of Microbiology, 2(2): 070-078.
13. Kang, S. M., Khan, A. L., Hussain, J., Ali, L., Kamran, M., Waqas, M., & Lee, I. J. (2012). Rhizonin A from Burkholderia sp. KCTC11096 and its growth promoting role in lettuce seed germination. Molecules, 17(7): 7980-7988.
14. Vetrano, F., Miceli, C., Angileri, V., Frangipane, B., Moncada, A., & Miceli, A. (2020). Effect of bacterial inoculum and fertigation management on nursery and field production of lettuce plants. Agronomy, 10(10): 1477.
15. Sottero, A. N., dos Santos Freitas, S., Tavares de Melo., A. M., & Trani, P. E. (2006). Rhizobacteria and lettuce: root colonization, plant growth promotion and biological control. Revista Brasileira de Ci\vec{e}ncia do Solo, 30(2).
16. Verrua, M1., Singh, A., Dwivedi, & Arora, N. K. (2020). Zinc and phosphate solubilizing Rhizobium radiobacter (LB2) for enhancing quality and yield of loose lea saline soil. Environmental Sustainability, 3: 209-218.
17. Ahmadi, N., Fatemi, H., Esmaielpour, B., & Soultani-Tolamod, A. A. (2020). Effect of bio-priming with plant growth promoting bacteria on growth and biochemical characteristics, phenol, flavonoid, vitamin C and nitrate in lettuce (Lactuca sativa L.) Rabicon cultivar in different growth substrates. Journal of Science and Technology of Greenhouse Culture, 11(2): fa41-fa58.
18. Kohler, J., Caravaca, F., Carcasco, L., & Roldan, A. (2006). Contribution of Pseudomonas mendocina and Glomus intraradices to aggregate stabilization and promotion of biological fertility in rhizosphere soil of lettuce plants under field conditions. Soil Use and Management, 22: 298-304.
19. Moncada, A., Vetrano, F., & Miceli, A. (2020). Alleviation of salt stress by plant growth-promoting bacteria in hydroponic leaf lettuce. Agronomy, 2020(10): 1523.
20. Aini, N., Yamika., W. S. D., & Ulum, B. (2019). Effect of nutrient concentration, PGPR and AMF on plant growth, yield and nutrient uptake of hydroponic lettuce. International Journal of Agriculture and Biology, 21: 175-183.
21. Utkhede, R. S., Lévesque, C. A., & Dinh, D. (2000). Pythium aphanidermatum root rot in hydroponically grown lettuce and the effect of chemical and biological agents on its control. Canadian Journal of Plant Pathology, 22:2: 138-144.
22. Sebring, R. L. Duiker, S. W. Berghage, R. D. Regan, J. M. Lambert, J. D. & Bryant, R. B. (2022). Gluconacetobacter diazotrophicus inoculation of two lettuce cultivars affects leaf and root growth under hydroponic conditions. Applied Sciences, 12: 1585.
23. Riser, E. C., Grabowski, J., & Glenn, E. P. (1984). Microbiology of hydroponically-grown lettuce. Journal of Food Protection, 47(10): 765-769.
24. De Almeida Moreira, V., da Silva Oliveira, C. E., Jalal, A., Bueno Gato, I. M., Sena Oliveira, T. J. S. S., Boleta, & Giolo, V. M. (2022). Inoculation with Trichoderma harzianum and Azospirillum brasilense increases nutrition and yield of hydroponic lettuce. Archives of Microbiology, 204: 440.
25. Ajijah, N., Apriyana, A. Y., Sriwuryandari, L., Priantoro, E. A., Janetasari, S. A., Pertiwi, T. Y. R., Suciati, A. M., Ardeniswan, & Sembiring, T. (2021). Beneficiary of nitrifying bacteria for enhancing lettuce (Lactuca sativa) and vetiver grass (Chrysopogon zizanioides L.) growths align with carp (Cyprinus carpio) cultivation in an aquaponic system. Environmental Science and Pollution Research, 28: 880-889.
26. Suzuki, W., Sugawara, M., Miwa, K., & Morikawa, M. (2014). Plant growth-promoting bacterium Acinetobacter calcoaceticus P23 increases the chlorophyll content of the monocot Lemna minor (duckweed) and the dicot Lactuca sativa (lettuce). Journal of Bioscience and Bioengineering, 118(1): 41-44.

1. A microbial inoculant for promoting leafy green plant growth comprising Rhodococcus erythropolis.

2. The microbial inoculant of claim 1, wherein the Rhodococcus erythropolis has a 98% nucleotide sequence similarity to the 16S region of SEQ ID NO:1.

3. The microbial inoculant of claim 1, also comprising Serratia grimesii.

4. A microbial inoculant for promoting leafy green plant growth comprising Serratia grimesii.

5. The microbial inoculant of claim 4, wherein the Serratia grimesii has a 98% nucleotide sequence similarity to the 16S region of SEQ ID NO:2.

6. The microbial inoculant of claim 5, also comprising Rhodococcus erythropolis with a 98% nucleotide sequence similarity to the 16S region of SEQ ID NO:1.

7. The microbial inoculant of claim 4, also comprising Pseudomonas toyotomiensis.

8. The microbial inoculant of claim 5, also comprising Pseudomonas toyotomiensis with a 98% nucleotide sequence similarity to the 16S region of SEQ ID NO:4.

9. A microbial inoculant for promoting leafy green plant growth comprising Stenotrophomonas maltophilia and Pseudomonas toyotomiensis.

10. The microbial inoculant of claim 9, wherein the Stenotrophomonas maltophilia has a 98% nucleotide sequence similarity to the 16S region of SEQ ID NO:3 and the Pseudomonas toyotomiensis has a 98% nucleotide sequence similarity to the 16S region of SEQ ID NO:4.

11. The microbial inoculant of claim 9 also comprising Rhodococcus erythropolis.

12. The microbial inoculant of claim 10 also comprising Rhodococcus erythropolis with a 98% nucleotide sequence similarity to the 16S region of SEQ ID NO:1.

13. The microbial inoculant of claim 11, also comprising Serratia grimesii.

14. The microbial inoculant of claim 12, also comprising Serratia grimesii with a 98% nucleotide sequence similarity to the 16S region of SEQ ID NO:2.

15. A microbial inoculant for promoting leafy green plant growth comprising Shinella zoogloeoides.

16. The microbial inoculant of claim 15, wherein the Shinella zoogloeoides has a 98% nucleotide sequence similarity to the 16S region of SEQ ID NO:5.

17. The microbial inoculant of claim 13, also comprising Shinella zoogloeoides.

18. The microbial inoculant of claim 14, also comprising Shinella zoogloeoides with a 98% nucleotide sequence similarity to the 16S region of SEQ ID NO:5.

19. The microbial inoculant of claim 1 in which the leafy green plant is Lactuca sativa.

20. A method for promoting plant growth comprising applying a composition of the microbial inoculant of claim 1 directly or through a water supply to the roots of the leafy green plants in an amount that provides for positive benefits relative to a control leafy green plant that does not receive an application of said microbial inoculant.


SEQUENCE LISTING (5 sequences)

SEQ ID NO: 1 - DNA, 1504 bp, Rhodococcus erythropolis
  source 1..1504: mol_type = genomic DNA
tttgatcctggctcaggacgaacgctggcggcgtgcttaacacatgcaagtcgagcggtaaggcctttcggggtacacgagcggcgaacgggtgagtaacacgtgggtgatctgccctgcacttcgggataagcctgggaaactgggtctaataccggatatgacctcctatcgcatggtgggtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgcaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggttccttccacggaatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagctgcagagatgtggccccccttgtggtcggtatacaggtggtgcatggctgtcgtcagctcgtgtcgtgagatgttgggttaagtcccgcaacgagcgcaacccctatcttatgttgccagcacgttatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcttaaccccttgtgggagggagccgtcgaaggtgggatcggcgattgggacgaagtcgtaacaaggtagccgtaccggaaggtgcggctggatcacctcc

SEQ ID NO: 2 - DNA, 1538 bp, Serratia grimesii
  source 1..1538: mol_type = genomic DNA
ttgaagagtttgatcatggctcagattgaacgctggcggcaggcctaacacatgcaagtcgagcggtagcacaagagagcttgctctctgggtgacgagcggcggacgggtgagtaatgtctgggaaactgcctgatggagggggataactactggaaacggtagctaataccgcataacgtcttcggaccaaagtgggggaccttcgggcctcacgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaagggtagtgtgttaatagcacattgcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaattcgctagagatagcttagtgccttcgggaactctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcgtatacaaagagaagcgaactcgcgagagcaagcggacctcataaagtacgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcct

SEQ ID NO: 3 - DNA, 1540 bp, Stenotrophomonas maltophilia
  source 1..1540: mol_type = genomic DNA
tgaagagtttgatcctggctcagagtgaacgctggcggtaggcctaacacatgcaagtcgaacggcagcacagaggagcttgctccttgggtggcgagtggcggacgggtgaggaatacatcggaatctactttttcgtgggggataacgtagggaaacttacgctaataccgcatacgacctacgggtgaaagcaggggatcttcggaccttgcgcgattgaatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaatccagccggctaatacctggttgggatgacggtacccaaagaataagcaccggctaacttcgtgccagcagccgcggtaatacgaagggtgcaagcgttactcggaattactgggcgtaaagcgtgcgtaggtggttatttaagtccgttgtgaaagccctgggctcaacctgggaactgcagtggatactggatgactagaatgtggtagagggtagcggaattcctggtgtagcagtgaaatgcgtagagatcaggaggaacatccatggcgaaggcagctacctggaccaacattgacactgaggcacgaaagcgtggggagcaaacaggattagataccctggtagtccacgccctaaacgatgcgaactggatgttgggtgcaatttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggccttgacatgtcgagaactttccagagatggatgggtgccttcgggaactcgaacacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacggccagggctacacacgtactacaatggtagggacagagggctgcaagccggcgacggtaagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacggtgtggccgatgactggggtgaagtcgtaacaaggtagccgtatcggaaggtgcggctggatcacctcctt

SEQ ID NO: 4 - DNA, 1531 bp, Pseudomonas toyotomiensis
  source 1..1531: mol_type = genomic DNA
tgaagagtttgatcatggctcagattgaacgctggcggcaggcctaacacatgcaagtcgagcggatgaggggagcttgctccctgatttagcggcggacgggtgagtaatgcctaggaatctgcctagtggtgggggataacgttccgaaaggaacgctaataccgcatacgtcctacgggagaaagcgggggatcttcggacctcgcgccattagatgagcctaggtcggattagctagttggtgaggtaaaggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggtattcacctaatacgtgagtattttgacgttaccgacagaataagcaccggctaacttcgtgccagcagccgcggtaatacgaagggtgcaagcgttaatcggaattactgggcgtaaagcgcgcgtaggtggttcgttaagttggatgtgaaagccccgggctcaacctgggaactgcatccaaaactggcgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggaactcagacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccttcggggggacggttaccacggagtgattcatgactggggtgaagtcgtaacaaggtagccgtaggggaacctgcggctggatcacctcct

SEQ ID NO: 5 - DNA, 1471 bp, Shinella zoogloeoides
  source 1..1471: mol_type = genomic DNA
tttgatcctggctcagaacgaacgctggcggcaggcttaacacatgcaagtcgaacgccccgcaaggggagtggcagacgggtgagtaacgcgtgggaatctacccatctctacggaataactcagggaaacttgtgctaataccgtatacgcccttcgggggaaagatttatcggagatggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggaattactgggcgtaaagcgcacgtaggcgggtatttaagtcaggggtgaaatcccagagctcaactctggaactgcctttgatactgggtacctagagtatggaagaggtaagtggaattccgagtgtagaggtgaaattcgtagatattcggaggaacaccagtggcgaaggcggcttactggtccattactgacgctgaggtgcgaaagcgtggggagcaaacaggattagataccctggtagtccacgccgtaaacgatgaatgttagccgtcggcatgcatgcatgtcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatgtcggtcgcggattacagagatgttttccttcagttaggctggaccgaacacaggtgctgcatggctgtcgtcagctcgtgtcgtgagatgttgggttaagtcccgcaacgagcgcaaccctcgcccttagttgccagcattcagttgggcactctaaggggactgccggtgataagccgagaggaaggtggggatgacgtcaagtcctcatggcccttacgggctgggctacacacgtgctacaatggtggtgacagtgggcagcgagacagcgatgtcgagctaatctccaaaagccatctcagttcggattgcactctgcaactcgagtgcatgaagttggaatcgctagtaatcgcggatcagcatgccgcggtgaatacgttcccgggccttgtacacaccgcccgtcacaccatgggagttggttttacccgaaggcactgcgctaaccgcaagggggcaggtgaccacggtagggtcagcgactggggtgaagtcgtaacaaggtagccgtaggggaacctgcggctggatcacctcc